(19) United States
(12) Patent Application Publication
(10) Pub. No.: US 2015/0034090 A1
(43) Pub. Date: Feb. 5, 2015

(54) CANNULA COVER AND ADJUSTER

(71) Applicants: Jacqueline Berger, Redhead (AU); Jeffrey John Julian, Burbank, CA (US)

(72) Inventors: Jacqueline Berger, Redhead (AU); Jeffrey John Julian, Burbank, CA (US)

(21) Appl. No.: 13/956,885

(22) Filed: Aug. 1, 2013

(51) Int. Cl. A61M 16/06

(52) U.S. Cl. CPC A61M 16/0683; A61M 16/0666
USPC 128/207.18

(57) ABSTRACT

A cannula cover fitted to a nasal cannula, such that the cover may be slid up and down the nasal cannula. In one aspect, when the cannula is in use, the cover may be slid to a position that contacts the user's chin, thus comfortably securing the cannula to the user. In another aspect, when the cannula is not in use, the cover may be slid up towards the nasal prongs covering them like a protective pocket.

### BACKGROUND

Patients requiring the use of a nasal cannula for delivery of oxygen or supplemental air often find that the cannula comes loose during activity or while sleeping due to a lack of a secure fit. Further, it is not uncommon for the nasal cannula to fall to the ground when it is removed to perform tasks such as changing clothes, using the restroom, bathing or attending to other needs, creating a situation that is unhygienic. It has been observed that hospitals and medical staff do not have a good protocol concerning nasal cannulas and, in particular, often do not have procedures in place to prevent the cannulas from contacting unsanitized surfaces. As a result, hair, dirt and other physical things sometimes become stuck to the cannulas, which may give rise to infections when they are later used.

While known nasal cannulas provide a plastic slide bolo to tighten the cannula during use, the slide bolos are often uncomfortable and provide no protection for the cannula when not in use.

Accordingly, there is a need for a nasal cannula that is comfortable to wear and fits securely during daily use. There is also a need for an improved nasal cannula in which the nasal prongs are protected from dirt, germs and other sources of disease and infection when not in use.

### SUMMARY

It is an object of the present invention to provide a cover and adjuster for a nasal cannula, making the cannula comfortable to wear and fit securely during daily use.

It is also an object of the present invention to provide a cover and adjuster for a nasal cannula in which the nasal prongs are protected from dirt, germs and other sources of disease and infection when not in use.

In one embodiment of the present invention, a soft, flexible cover is fitted to a nasal cannula, such that the cover may be slid up and down the nasal cannula. In one aspect, when the cannula is in use, the cover may be slid to a position that contacts the user's chin, thus comfortably securing the cannula to the user. In another aspect, when the cannula is not in use, the cover may be slid up towards the nasal prongs covering them like a protective pocket and providing a barrier from dirt and germs.

The above cover may be in the form of a flexible funnel, being narrow at the bottom and wider at the top. The narrow bottom cinches the cannula and keeps the cover in position. The wider top provides a comfortable surface for the user when cannula is in use and receives the nasal prongs when it is not in use. The nasal cannula thus configured with the cover enables a freedom of movement during use. Also, with a simple pull of the cover towards the nasal prongs the flexible pocket covers the prongs enveloping them in the protective case. The cover may include a closure mechanism for the formed pocket, such as a zipper, snaps, buttons, or other closure mechanisms. In another aspect, the cover can be removed and replaced as needed, either for cleaning or replacement.

The nasal cannula cover may be used for all types of patients, including infants, children, youths, adults, women, men, and the elderly. Also, any suitable fabric may be used, including, but not limited to, Neoprene, rubber, and other fabrics and materials. In addition, the covering may be in different suitable shapes and forms.

### BRIEF DESCRIPTION OF THE DRAWINGS

FIG. 1 is an illustration of a nasal cannula and cover in accordance with an embodiment of the present invention.

FIG. 2 is an illustration of the nasal cannula being cinched part way up by the cannula cover, as may be the case when the nasal cannula is being used.

FIG. 3 is an illustration of the cannula cover positioned to encapsulate the nasal prongs.

FIG. 4 illustrates one of many possible patterns for the cannula cover.

FIG. 5 is a close up view of a cannula cover positioned to encapsulate the nasal prongs and showing the cover's overall construction and stitching.

FIG. 6 also illustrates the positioning of the cannula cover along the nasal cannula.

FIG. 7 is a side view of the cannula and cannula cover being worn by a user.

FIG. 8 is a front view of the cannula and cannula cover being worn by a user.

### DETAILED DESCRIPTION

The cannula cover in accordance with the present invention is preferably intuitive to use and provides protection for the prongs of a nasal cannula when the cannula not in use. The cover is designed to be pulled up over the nasal prongs encapsulating the prongs and shielding them from coming into contact with bacteria, viruses and pathogens, and or staph and the like. Also, the cover acts as a comfortable neck cinch, improving fit and comfort while the wearer is using the cannula. The cover can be removed and replaced as needed either for cleaning or replacement. The cover may be configured as an after-market or add-on product for use with an existing or separate nasal cannula. The cover may also come in a variety of colors and styles to suit the preferences of the user and possibly to help users feel less self-conscious when out in public. Added benefits of using this device is shorter hospital stays due to the avoidance of possible infections caused by contact with dirty cannulas, ultimately providing a financial benefit to the patient, their families, as well the medical and insurance industries, and dramatically reducing the pain and suffering related to nosocomial infections.

The cannula cover is, in one embodiment, a sterile encasement made to fit on basically any size nasal cannula found on the market. The cannula cover is designed to improve the comfort and fit of a nasal cannula while the cannula is in use, reducing prolonged rubbing of the skin, discomfort and unexpected cannula dislodgement.

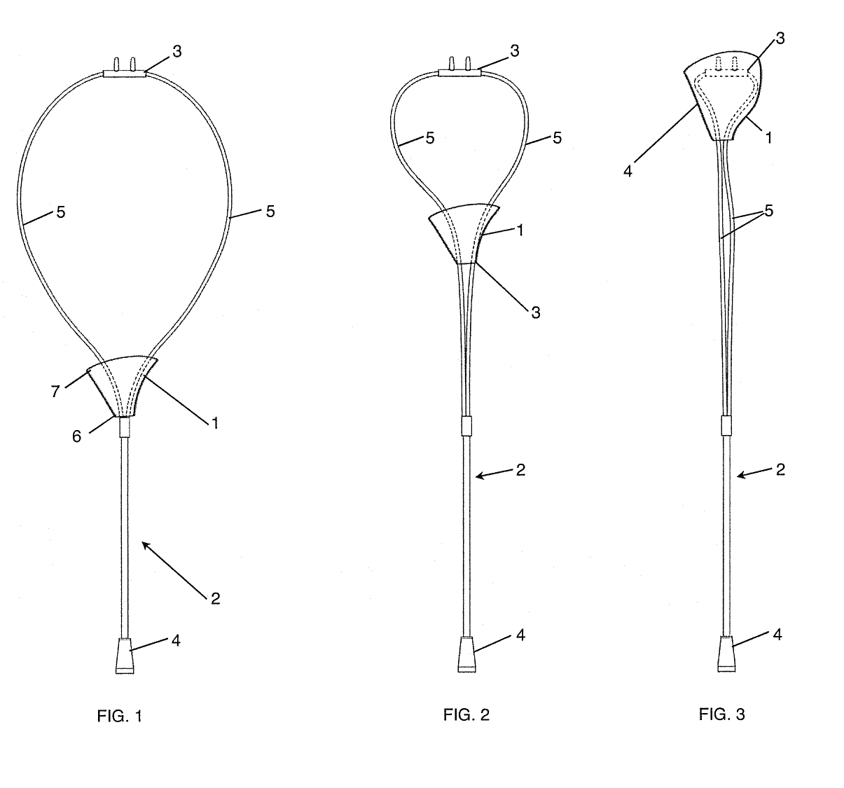

FIG. 1 illustrates a nasal cannula and cover in accordance with an embodiment of the present invention. A typical nasal cannula 2 includes nasal cannula prongs 3 and external connector 4, which are connected to each other by tubes 5. External connector 4 is typically connected to an oxygen or air supply. Cannula cover 1 is configured to slidably move over the nasal cannula. It is preferably made of an elastic or stretchable material which is generally funnel-shaped, though other shapes would be equally suitable for the present invention. Cannula cover 1 has a small opening 6 on one end and a larger opening 7 at the other end. Small opening 6 is capable of cinching the tubes 5 as the cover is slid up the cannula towards the nasal prongs 3. Larger opening 7 is sized and shaped so that it feels comfortable to the user when the cover is slid up to the user's chin during use. Openings 6 and 7 may be sized so that the nasal cannula can be threaded through the cannula cover, enabling the cannula cover to be removed for cleaning and/or replacement.

FIG. 2 illustrates the cannula cover 1 slid to an intermediate position along the nasal cannula, as may be the case when the nasal cannula is in use.

FIG. 3 illustrates the cannula cover 1 slid to a position that encompasses nasal prongs 3.

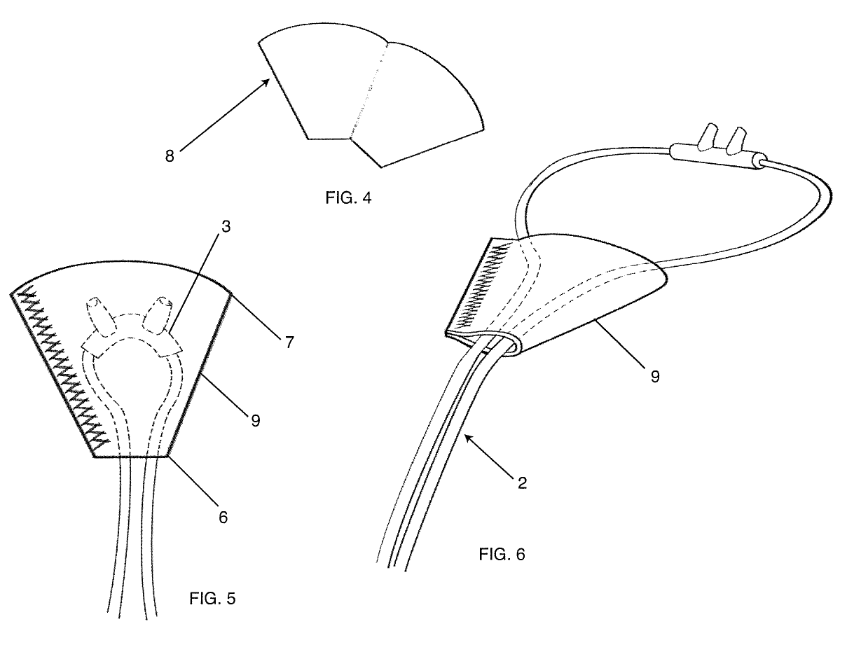

FIG. 4 illustrates one of many possible patterns 8 for the cannula cover. The illustrated pattern defines a cover having a generally flattened, funnel-shaped form, as depicted in FIG. 6. Alternatively, the pattern may define a cover having other suitable shapes and forms. Generally, any suitable fabric may be used, including, but not limited to, Neoprene, rubber, and other fabrics and materials. Preferably, the pattern and material should be safe and flexible and permit the cover to slide along the nasal cannula, reasonably retain the position to which its set, and be able to envelop the nasal prongs.

FIG. 5 is a close up view of a cannula cover 9 positioned to encapsulate the nasal prongs 3 and showing the cover's overall construction and stitching. In the figure, cannula cover 9 is formed by a folded material, such as Neoprene or another suitable material, that has been stitched. However, any number of closing methods may be used, including but not limited to, stitching, adhesives, sonic or other welding, tape, snaps, and Velcro.

In one embodiment, top end 7 of cannula cover 9 is loose, allowing the nasal prongs 3 to fit easily into the cover with a simple pulling of the cannula in the opposite direction from the cover. Also, bottom end 6 of cannula cover 9 is tight around the nasal cannula, making a tight friction fit to secure the cover at a desired position along the cannula without slipping.

FIG. 6 similarly illustrates the positioning of the cannula cover 9 along the nasal cannula 2. Again, the bottom end of the cover is a small snug opening enabling the elastomer of the cover to act as a tight friction fit, retaining the desired position without slipping out of place. The top end of the cover is a large opening enabling the cover to act as a funnel allowing the cannula to expand for a comfortable fit. This enables the user to move normally while wearing the cannula while avoiding or lessening the problem of having the cannula dislodge.

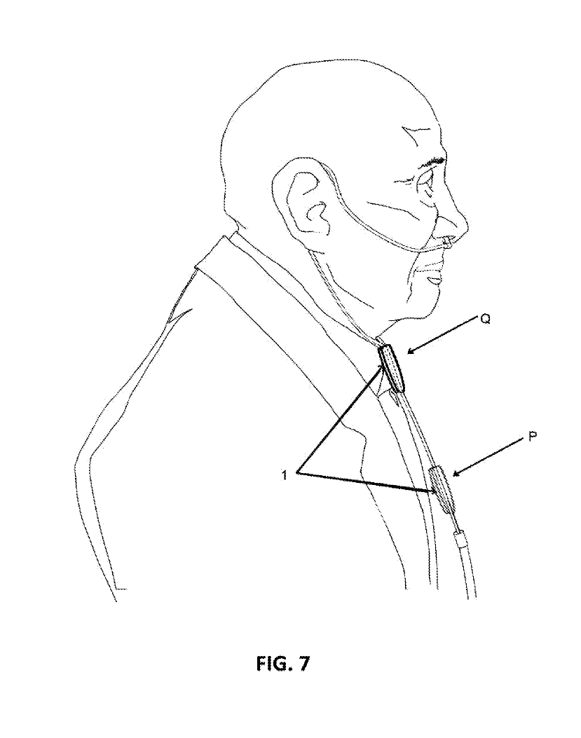

FIG. 7 is a side view of the cannula 2 with cannula cover 1 being worn by a user. It illustrates how the cannula cover 1 can be slid along the cannula from a position P to a position Q. As illustrated, the cover, acting as a cinch, can be moved up and down, providing a secure and comfortable fit.

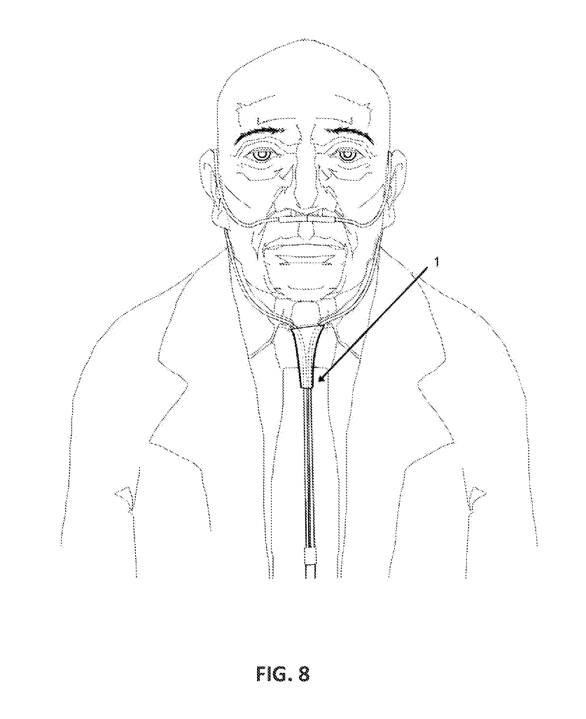

FIG. 8 is a front view of the cannula 2 and cannula cover 1 being worn by a user.

The cannula cover as described above is readily adapted as an after-market or add-on product for use with nasal cannulas having a wide variety of sizes and designs.

The illustrative descriptions of the principles of the present invention are to enable any person skilled in the art to make or use the disclosed invention. These descriptions are susceptible to numerous modifications and alternative arrangements by those skilled in the art. Such modifications and alternative arrangement are not intended to be outside the scope of the present invention. The appended claims are intended to cover such modifications and arrangements. Thus, the present invention should not be limited to the described embodiments but, instead, is to be accorded the broadest scope consistent with the principles and novel features disclosed herein.

What is claimed is:

1. A cover for a nasal cannula comprising:
a material formed into a roughly funnel-like shape having a lower edge and an upper edge, the material capable of slidable engagement with a nasal cannula;
the lower edge of the material capable of cinching the nasal cannula and causing frictional contact with the nasal cannula; and
the upper edge of the material capable of sliding over nasal prongs of the nasal cannula; and
the material sized to encompass the nasal prongs when the material is slid over the nasal prongs.

2. The cover of claim 1 wherein the material is elastic or stretchable.

3. The cover of claim 1 wherein the material is Neoprene.

4. The cover of claim 1 wherein the material is generally flat and is formed into a roughly funnel-like shape by joining opposite sides of the material.

5. The cover of claim 4 wherein the oppositions sides of the material are joined by one or more of stitching, adhesives, sonic or other welding, tape, snaps, and/or Velcro.
